(19) United States
(12) Patent Application Publication
(10) Pub. No.: US 2007/0182729 A1
(43) Pub. Date: Aug. 9, 2007

(54) Apparatus for interventional therapy

(76) Inventors: Klaus Klingenbeck-Regn, Nurnberg (DE); Judith Regn, Nurnberg (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/657,914

(22) Filed: Jan. 25, 2007

(30) Foreign Application Priority Data
Jan. 25, 2006 (DE) 10 2006 003 610.7

(51) Int. Cl. G06T 1/00; A61B 5/00; A61B 17/00

(52) U.S. Cl. 345/418; 600/300; 606/1

(57) ABSTRACT

The invention relates to a system for performing an interventional therapy of a patient comprising a display unit. The display unit is assigned as a single large format flat screen display, on which information relating to monitoring of the medical intervention, patient information and information to control the system are able to be presented.

### CROSS REFERENCE TO RELATED APPLICATIONS

This application claims priority of German application No. 10 2006 003 610.7 filed Jan. 25, 2006, which is incorporated by reference herein in its entirety.

### FIELD OF THE INVENTION

The invention relates to an apparatus for interventional patient therapy, with a display unit, on which information relating to monitoring of a medical intervention can be presented.

### BACKGROUND OF THE INVENTION

These types of apparatus are generally known for interventional therapy. The known apparatuses are provided with imaging components which allow the progress of the intervention to be monitored. The imaging components create image information which can be displayed on a plurality of individual monitors. The monitors concerned are usually cathode ray tube monitors, which are replaced in more recent apparatuses by flat screen displays, so that the display unit weighs less and occupies less space.

An example of such an apparatus is a monoplanar or biplanar x-ray system for monitoring an intervention undertaken with the aid of a catheter. The image information for monitoring the movement of the catheter is displayed on displays screens which are held on a ceiling-mounted support.

One disadvantage of the known apparatuses is their limited handling flexibility. As a rule a monitor is assigned in each case to an imaging component and displays the image information created by the imaging component. Thus it is generally not possible to simply display any image information on a specific monitor. In addition there is restricted space available on the individual monitor for displaying image information. It is thus not easily possible to display additional information on a specific monitor or to enlarge the display.

### SUMMARY OF THE INVENTION

Using this prior art as its starting point, the underlying object of the invention is to create an apparatus for interventional therapy of which the display unit provides a greater degree of flexibility in the presentation of image information.

This object is achieved by an apparatus with the features of the independent claim. Advantageous embodiments and developments are specified in its dependent claims.

In the apparatus for interventional patient therapy the display unit comprises a single screen providing the option of presenting information from different information sources at a position able to be selected by a user. The use of a single display screen of a suitable size gives users the opportunity of displaying the information that they need in the form that they need without as a rule being subject to any space restrictions. In addition users can also freely select the arrangement of the image information on the common display screen. The apparatus thus provides an increased degree of flexibility with regard to the presentation of the image information.

In a preferred embodiment, a form of presentation selected by a user can be stored under a code and can be retrieved again by the user as required by referencing this code. This offers the advantage that the forms of presentation necessary for the intervention are selected in advance and are able to be rapidly retrieved quickly during the intervention.

In a further preferred embodiment, a form of presentation can be set using information from the information sources. Preferably the information for setting the form of presentation is linked to the information to be presented. In this way the forms of presentation required for presentation of the information can be set automatically.

The display screen concerned can be a touch-sensitive screen. This enables users to influence the display on the display screen without further input devices.

In addition it is possible to provide an input option with the aid of a remote control. This input option is especially of advantage if the user responsible for how the information is presented on the display screen has frequently to change the location at which he or she is operating during an intervention.

### BRIEF DESCRIPTION OF THE DRAWINGS

Further advantages and details of the invention can be found in the description below, in which exemplary embodiments of the invention are explained in detail with reference to the enclosed drawings. The Figures show:

FIG. 1 an apparatus for the magnetic field-supported catheter intervention, which is equipped with a single screen for presentation of the image information necessary to monitor the intervention;

FIG. 2 a view of the display surface of the display screen of the apparatus from FIG. 1; and

FIG. 3 a block diagram of the apparatus depicted in FIG. 1.

### DETAILED DESCRIPTION OF THE INVENTION

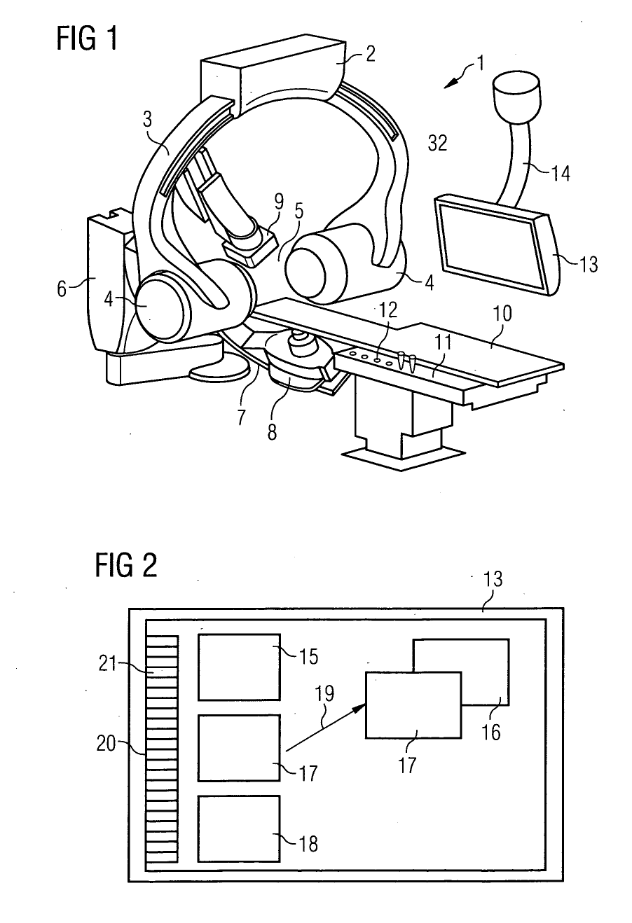

FIG. 1 shows a system 1 for magnetic field-supported catheter intervention. The system 1 comprises a C-arm 3 mounted on a ceiling support 2, with magnets 4 accommodated at either end of the C-arm, which create a magnetic field in the space therebetween 5, through which a magnetic tip of a catheter can be navigated in the body of a patient to be treated. To monitor the movement of the catheter in the body of the patient a further C-arm 7 accommodated on a stand 6 is provided, with an x-ray source 8 and an x-ray detector 9 being accommodated at the ends of said further C-arm.

During the catheter intervention a patient to be treated lies on a patient table 10, into which is also integrated a control desk 11 with a plurality of control elements 12, such as a keyboard or joystick for example. The control desk 11 is used for control of the C-arms 3 and 7 as well as of further components not shown in FIG. 1, for example a drive element which moves the catheter in the body of the patient to be examined.

A single flat screen display 13 is arranged over the patient table 10 in the area of the control desk 11, on which the information required for operation of the system 1 is displayed. The flat screen display 13 is mounted with the aid of a support 14 on the ceiling which allows it to be tilted and swiveled.

The flat screen display 13 is preferably a flat screen display with a resolution of at least 1200×1600 pixels. With this level of resolution the flat screen provides sufficient room to display the information content of four conventional display screens with a resolution of 600×800 pixels.

The flat screen display 13 is further preferably a color display screen or a color display screen able to be switched between a black and white display mode and a color display mode.

FIG. 2 shows a view of the display area of the flat screen display 13. The flat screen display 13 can display different windows 15 to 18, of which for example the window 15 shows current x-ray images of the x-ray detector 9. In a further window 16 information can be displayed about examinations which have already been conducted prior to the intervention. Examples of this are two-dimensional or three three-dimensional images recorded with the aid of computer tomography devices or magnetic resonance devices. Ultrasound recordings can also be displayed in the window 16. A further window 17 contains information about the current bodily functions of the patient to be examined, for example current electrocardiograms, with which the patient to be examined is monitored. A further window 18 can for example contain data from the database of an HIS (=Hospital Information System), an RIS (=Radiology Information System) or a CIS (=Cardiology Information System).

The windows 15 to 18 can be moved around by the user in the display area of the flat screen display 13 in any desired manner. Furthermore the sizes of windows 15 to 18 can be reduced or increased as required. For example the window 17 can be moved in the direction of an arrow 19 to create space for enlarging the window 15.

Furthermore a menu bar 20 can be provided. Different shortcuts for different forms of presentation can be stored by users in the menu bar 20, so that users can activate a menu entry 21 to call up a specific form of presentation. Preferably the different forms of presentation are stored before the start of the intervention, so that the different forms of presentation can be retrieved at short notice.

Forms of presentation in this case should be understood as being a specific configuration or parameterization of the windows 15 to 18 as regards arrangement, resolution, size, color display, modality and display mode of the windows. Display mode in this case should be understood as the behavior of the window in relation to other windows. In particular specific windows with important information can always be placed in the foreground. Modality is further to be understood as the behavior of the window in respect of the application creating it. Modal windows do not allow further processing with the application which has created them until they have been closed.

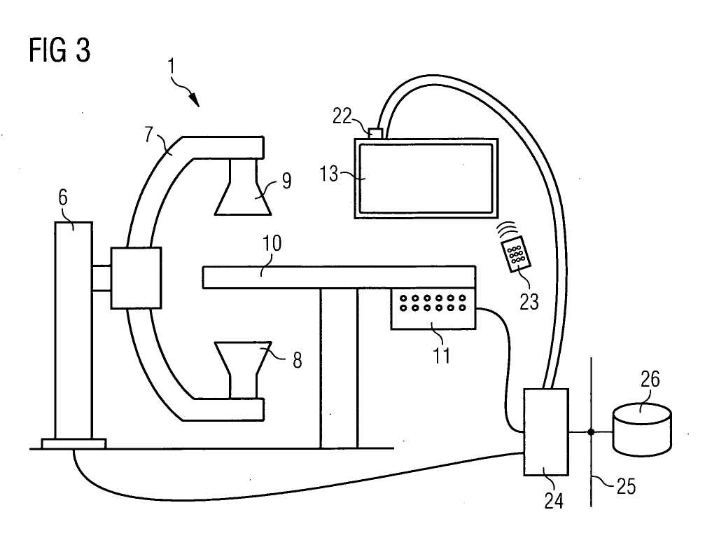

FIG. 3 shows a block diagram of the system 1. To aid clarity, the block diagram in FIG. 3 only shows the C-arm 7 together with its stand 6. The C-arm 3 with the magnets 4 is by contrast not shown in FIG. 3.

In accordance with FIG. 3 the flat screen display 13 is equipped with an infrared sensor 22, via which the flat screen display 13 can be operated with the aid of a remote control 23. This embodiment of the system 1 is especially of advantage, if the user responsible for the display on the flat screen display 13 has to operate at changing locations.

In addition it is possible to control the display of the flat screen display 13 with the aid of the control desk 11. Suitable keys can be provided for this purpose on the control desk 11, which the user can program in advance to assign different forms of presentation to them. The individual forms of presentation can however also be called up with the aid of a mouse pointer shown on the flat screen display 13, the movement of which is controlled by a control means embodied at the control desk 11. The desired menu entry 21 can then be selected and activated for example with the aid of the mouse pointer.

Furthermore it is possible to equip the flat screen display 13 with the option of touch-screen input. In this case users can call up individual forms of presentation by touching the flat screen display 13 or move or resize individual windows 15 to 18.

In a further modified embodiment the flat screen display 13 is controlled with the aid of a laser pointer. For this purpose a transparent disk can be arranged in front of the flat screen display 13. This disk can be used to define the point at which the laser beam emitted by the laser pointer hits the flat screen display 13. The laser beam of the laser pointer can then perform the function of a mouse pointer. A menu entry 21 to be selected in the menu bar 20 can then be activated accordingly by the laser pointer.

In accordance with FIG. 3 the control desk 11 and the flat screen display 13 as well as the infrared sensor 22 are connected to a system processor 24 which also controls the movement of the C-arm 3 with the magnet 4 as well as the movement of the C-arm 7 with the x-ray source 8 and the x-ray detector 9. The system processor 24 can be connected to a network 25, and by accessing a data memory 26, for example a PACS server, can retrieve image representations of pre-interventional examinations for display on the flat screen display 13. In addition data from an HIS, RIS or a CIS can also be retrieved from the measurement data memory 26. The data retrieved from the HIS, RIS or CIS can be presentational image data or data available in text form.

Data which can be retrieved from the data memory 26 can comprise presentation data which allows automatic selection of a suitable form of presentation. For example x-ray images retrieved from an RIS can contain presentation data or be linked to presentation data which allows a suitable presentation for the x-ray image to be selected automatically. This enables the burden on the medical personnel to be greatly reduced when they are carrying out the intervention.

It should be pointed out that large-format flat screen displays 13 for presentation of patient or system information can also be used with further systems for interventional medicine. In particular these types of large-format flat screen displays 13 can replace a plurality of monitors usually employed for control of monoplanar and biplanar C-arm systems.

1-14. (canceled)

15. An apparatus for performing an interventional medical procedure of a patient, comprising:
a device that monitors the interventional medical procedure; and
a display unit comprising a display screen that presents the monitoring information and a further information if required by a user from an information source, wherein the monitoring information and the further information are presented on the display screen at a position selected by the user.

16. The apparatus as claimed in claim 15, wherein the further information comprises information selected from the group consisting of: a medical procedure of the patient conducted prior to the current interventional medical procedure, a current physiological monitoring signal of the patient, and patient data from a database.

17. The apparatus as claimed in claim 16, wherein the database is selected from the group consisting of: hospital information system, radiology information system, and cardiology information system.

18. The apparatus as claimed in claim 15, wherein the information is presented according to a form of presentation.

19. The apparatus as claimed in claim 18, wherein the form of presentation is selected by the user and is stored under an assigned code and retrieved again by the user with the code.

20. The apparatus as claimed in claim 19, wherein the code is inputted as a menu entry in a menu bar that is displayed on the display screen.

21. The apparatus as claimed in claim 18, wherein the form of presentation is set using the further information from the information source.

22. The apparatus as claimed in claim 21, wherein the further information to be presented from the information source includes the form of presentation.

23. The apparatus as claimed in claim 15, wherein the display screen displays a window linked to the information source.

24. The apparatus as claimed in claim 23, wherein the window is moved on the display screen.

25. The apparatus as claimed in claim 23, wherein a size of the window is varied.

26. The apparatus as claimed in claim 15, wherein the display screen comprises a touch screen input.

27. The apparatus as claimed in claim 15, wherein the display screen is controlled by a remote control.

28. The apparatus as claimed in claim 27, wherein the remote control is a laser pointer.

29. The apparatus as claimed in claim 27, wherein the remote control is an infrared remote control.

30. The apparatus as claimed in claim 15, wherein the display screen is a flat screen display.

31. The apparatus as claimed in claim 30, wherein the flat screen display has a resolution of 1200×1600 pixels.

32. The apparatus as claimed in claim 30, wherein the flat screen display is mounted so that it can be tilted and swiveled on a support.

33. The apparatus as claimed in claim 15, wherein the display screen is connected to a system processor and the system processor is connected to a network that accesses a data memory comprising the further information.

34. A method for presenting information during an interventional medical procedure of a patient, comprising:
motoring the interventional medical procedure; and
presenting the monitoring information and a further information if required by a user from an information source on a display unit, wherein the monitoring information and the further information are presented on the display unit at a position selected by the user.
